(19) United States
(12) Patent Application Publication
(10) Pub. No.: US 2024/0036066 A1
(43) Pub. Date: Feb. 1, 2024

(54) METHOD FOR MONITORING AND ADJUSTING REAGENT FOR HEMATOLOGY

(71) Applicant: Radiometer Medical ApS, Brønshøj (DK)

(72) Inventors: Thomas Steen HANSEN, Brønshøj (DK); Heine HANSEN, Brønshøj (DK); Melania Andrea BURKHARDT, Brønshøj (DK); Willy Lindegaard ANDERSEN, Brønshøj (DK)

(21) Appl. No.: 18/258,398

(22) PCT Filed: Dec. 22, 2021
(86) PCT No.: PCT/EP2021/087410

§ 371 Date: Jun. 20, 2023

(30) Foreign Application Priority Data
Dec. 22, 2020 (EP) 20216585.8

(51) Int. Cl. G01N 33/96; G01N 35/10; G01N 1/34

(52) U.S. Cl. CPC G01N 33/96; G01N 35/1002; G01N 1/34

(57) ABSTRACT

A method of preparation of a blood sample with a reagent for hematology is disclosed. The method comprises combining a blood sample with an initial volume of a first reagent for provision of an initial mixture, monitoring the initial mixture, wherein monitoring the initial mixture comprises obtaining a first blood parameter of a first blood component of the initial mixture, determining a first combination scheme based on the first blood parameter, and adding a first volume of the first reagent and/or a first volume of a second reagent to the initial mixture according to the first combination scheme for provision of a first mixture

The present disclosure relates generally to improvements of hematology analysis, such as for patient care.

### BACKGROUND

Hematology is a branch of medicine covering diseases related to the blood and its components, including methods of treatment, diagnosis, analysis, etc. Hematology encompasses a number of different assessments that can be performed on blood and/or components of the blood. One or more of the assessments could require preparation of a blood sample prior to the actual assessment.

Typical hematology analysis is performed in a laboratory setting, and requires transportation of a blood sample to the laboratory. Thus, there is significant time delay between receiving a blood sample and providing analysis, which can slow down necessary patient care.

### SUMMARY

Accordingly, there is a need for accurate and fast hematology analysis in order to quickly assess and analyze components of a patient's blood. In particular, there is a need for accurate and fast point-of-care hematology analysis.

Disclosed herein is a method of preparation of a blood sample with a reagent for hematology, the method comprising combining a blood sample with an initial volume of a first reagent for provision of an initial mixture, monitoring the initial mixture, wherein monitoring the initial mixture comprises obtaining a first blood parameter of a first blood component of the initial mixture, determining a first combination scheme based on the first blood parameter, and adding a first volume of the first reagent and/or a first volume of a second reagent to the initial mixture according to the first combination scheme for provision of a first mixture.

Also disclosed herein is a method of preparation of a blood sample with a reagent for hematology, the method comprising combining or preparing a blood sample with an initial volume of a first reagent, e.g. according to an initial combination scheme, for provision of an initial mixture, monitoring the initial mixture, wherein monitoring the initial mixture comprises obtaining a first blood parameter of a first blood component of the initial mixture, determining a first combination scheme based on the first blood parameter, and preparing the initial mixture according to the first combination scheme for provision of a first mixture. Preparing the initial mixture according to the first combination scheme for provision of a first mixture optionally comprises one or more of adding a first volume of the first reagent, adding a first volume of a second reagent to the initial mixture, mixing the initial mixture, and incubating the initial mixture according to the first combination scheme for provision of a first mixture. Preparing the blood sample according to the initial combination scheme optionally comprises one or more of adding an initial volume of the first reagent, adding an initial volume of a second reagent to the blood sample, mixing the blood sample and reagent, and incubating the blood sample and reagent according to the initial combination scheme for provision of the initial mixture.

Further disclosed herein is a system for monitoring and adjusting a reagent for point of care hematology, the system comprising a controller, a reservoir for accommodating a reagent; and a cell measurement component for accommodating a blood sample, wherein the system is configured to perform the method as described herein.

It is an important advantage of the present disclosure to be able to monitor and adjust reagent levels to properly prepare blood samples prior to a full analysis for determining certain hematological parameters. This can improve the preparation of the blood samples and allow for minor adjustments to be made during the preparation process. Additionally, the present disclosure can avoid failed testing due to improperly prepared blood samples, which can significantly delay results and thus patient care. Accordingly, it is an important advantage to provide accurate final hematological results based on a blood sample.

Further, it is an important advantage to provide point-of-care hematological results, rather than having to transport the blood to a laboratory setting. Moreover, it is an important advantage of the present disclosure to be able to reduce changing consumables.

The present disclosure allows for improved preparation of blood samples for hematological analysis, in particular though the use of monitoring and adjusting a proper dosing of reagent to the blood sample. This can further improve the final results of the analysis, such as providing an accurate complete blood cell count. Further, advantageously the preparation, and eventual analysis, can all be done at a point-of-care facility, such as an emergency room. Additionally, the present disclosure allows for low mixing ratios of blood to reagent as compared to traditional ratios of about 1:10000 to 1:50000. Thus, the disclosure can reduce the need for changing consumables, enabling a smaller footprint.

### BRIEF DESCRIPTION OF THE DRAWINGS

The above and other features and advantages of the present disclosure will become readily apparent to those skilled in the art by the following detailed description of exemplary embodiments thereof with reference to the attached drawings, in which:

FIG. 1 illustrates a method as disclosed herein,

FIG. 2 illustrates a process as disclosed herein,

FIGS. 3A-3B illustrate a schematic of a system utilizing methods disclosed herein, and

FIG. 4 illustrates a schematic of a system utilizing methods disclosed herein.

### DETAILED DESCRIPTION

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

A method of monitoring and adjusting a reagent for a blood sample is disclosed. The method can be used to prepare a blood sample for hematology, e.g., blood analysis, blood component analysis, hematology analysis, hematological analysis, hematological assessment. In particular, the method can be used to prepare a blood sample for white blood cell differential (WBC DIFF), or white blood cell count, e.g. including 3-part or 5-part white blood cell differential, or white blood cell count. In particular, the method can be used to prepare a blood sample for complete-blood-counting (CBC), e.g. including 3-part or 5-part WBC DIFF. As discussed herein, CBC may include a WBC DIFF, or the terms may be used interchangeably. Other components of CBC analysis may be used as well, such as for the measurement, e.g. counting, of platelets. The blood sample can be from a patient. The blood sample can be from a human or animal.

In one or more exemplary methods, the blood sample is prepared prior to hematology analysis. For example, reagent may be added into the blood sample and/or the blood sample may be added into the reagent. As will be discussed, this may occur once. Alternatively, this may occur multiple times. The reagent may lyse and/or stain components of the blood sample. Further, mixing could be performed to the blood sample and/or any mixture of the blood sample and reagent. Also, incubation could be performed on the blood sample and/or any mixture of the blood sample and reagent. In one or more exemplary methods, pH of the blood sample can be adjusted. Other preparations could be performed as well, and the disclosure should not be so limited. Certain preparations are discussed as examples in more detail below.

Typically, preparations are designed for “standard” (e.g., normal) blood samples having particular properties (e.g., lipid/blood cell/platelet concentration, pH). However, more often than not the blood sample may not fit within the “standard” properties for one or more reasons, for instance, due to hereditary or environmental conditions. For example, the blood sample may have higher or lower red blood cell count outside of the “standard”, higher or lower cholesterol or other lipid content outside of the “standard”, among other parameters.

Accordingly, it may be advantageous to adjust the preparation of the blood sample prior to any hematology in order to properly prepare the non-standard blood sample to achieve accurate hematology assessment (e.g., vary a combination scheme), such as by monitoring and adjusting reagent levels. It can be particularly advantageous to be able to adjust the amount of reagent added to a blood sample, multiple times if necessary, in order to achieve an improved hematology analysis.

In particular, it may be advantageous to adjust a mixture of the blood sample with the reagent prior to any hematology in order to properly prepare the non-standard blood sample to achieve accurate hematology assessment (e.g., vary a combination scheme). The combination of blood sample with reagent will be called a mixture, e.g., solution, combination, herein. The mixture may be formed prior to any flow of the blood sample and the reagent through a mixture.

In addition, by determining the first combination scheme based on the first blood parameter of the initial mixture, the initial volume of the first reagent can be allowed to be minimized because any deficit of reagent in the initial volume may be compensated by the addition of the first and/or second reagent. Accordingly, no excess reagent may be required to prepare the initial mixture and the volume of reagent used may be able to be minimized. This can allow for an increased number of hematology assessments per volume of reagent and vice versa.

If the standard preparation was used for a non-standard blood sample, accurate hematology assessment may not be achieved. By properly preparing the mixture early in the process, the number of wasted samples may be reduced, along with the reduction of preparations needed during the procedure. Further, incorrect sample composition might also make measurements fail for replicates.

For example, the hematology can analyze one or more parameters of the blood sample. In one or more exemplary methods, the method of preparing a blood sample can be used for a complete blood count (CBC) analysis. In one or more exemplary methods, the method of preparing a blood sample can be used for a basic metabolic panel (BMP) analysis.

Advantageously, the disclosed methods can be used for point-of-care hematology testing, e.g. at the time and place of patient care. Thus, the method can be performed quickly and easily, such as in a patient's room or in an emergency room. In one or more exemplary methods, a blood sample can be taken directly from the patient to a system within the same room. For example, the method can be performed in a diagnostic room and/or testing room and/or hospital room and/or surgery room and/or operating room. Accordingly, blood sample can be taken from a patient and tested in the same area without transporting the blood sample. This can advantageously lead to the avoidance of transportation mix-ups or damage to the blood sample. Further, the methods can provide for significantly faster hematological analysis and results, thereby leading to improved patient care. Thus, the blood sample need not be taken to a laboratory, and the preparation and analysis can be performed in a single room, such as with the patient in it.

Alternatively or additionally, the disclosed methods may be used outside of point-of care hematology testing, such as laboratory hematology testing. Thus, the blood sample may be sent away from the patient.

Generally, one or more exemplary methods of the disclosure can be configured to properly prepare, e.g., treat, modify, pretreat, a blood sample for hematological testing, e.g. analysis, assessment. Specifically, the methods may be configured to prepare a blood sample with a reagent. The blood sample may be taken directly from a patient. The blood sample may be from a storage. The blood sample may be an aspirated blood sample. The blood sample discussed herein may be understood as an aspirated blood sample.

Different combination schemes can be used based on ascertained blood parameter(s) of the blood sample, such as in a mixture of the blood sample and reagent. It will be understood that the blood parameters may be taken from a combined mixture of blood and reagent. The blood parameter may be indicative of the blood sample. The blood parameter may be indicative of the mixture. The blood parameter may be indicative of a blood component of the mixture. The blood parameter may be indicative of a blood component of the blood sample.

As discussed throughout, a combination scheme may be a mixing scheme and/or a preparation scheme and/or a composition scheme and/or a composing scheme. The terms may be used interchangeably. The combination scheme can be used to mix and/or combine and/or incubate two components, for example two liquid components, for example a blood sample and a first reagent and/or a second reagent. The combination scheme may be used for partially and/or fully preparing a blood sample for hematological analysis, for example CBC analysis. Thus, the combination scheme may define a preparation of a blood sample via mixing and/or combining and/or incubating.

In one or more exemplary methods, a combination scheme, such as the first combination scheme and/or a second combination scheme, can vary based on red blood cell count of the initial mixture/first mixture/blood sample. In one or more exemplary methods, a combination scheme, such as the first combination scheme and/or a second combination scheme, can vary based on cholesterol content/count of the initial mixture/first mixture/blood sample. In one or more exemplary methods, a combination scheme, such as the first combination scheme and/or a second combination scheme, can vary based on white blood cell count of the initial mixture/first mixture/blood sample. In one or more exemplary methods, a combination scheme, such as the first combination scheme and/or a second combination scheme, can vary based on lipid content/count of the initial mixture/first mixture/blood sample. In one or more exemplary methods, a combination scheme, such as the first combination scheme and/or a second combination scheme, can vary based on platelet count of the initial mixture/first mixture/blood sample. In one or more exemplary methods, a combination scheme, such as the first combination scheme and/or a second combination scheme, can vary based on pH of the blood sample. In one or more exemplary methods, a combination scheme, such as the first combination scheme and/or a second combination scheme, can vary based on an electrical property, such as impedance, e.g. resistance and/or capacitance/inductance, and/or conductivity, e.g. at one or more frequencies, of the initial mixture/first mixture/blood sample. In one or more exemplary methods, a combination scheme, such as the first combination scheme and/or a second combination scheme, can vary based on an optical property, such as turbidity, absorbance and/or transmittance, e.g. at one or more wavelengths, of the initial mixture/first mixture/blood sample. In one or more exemplary methods, a combination scheme, such as the first combination scheme and/or a second combination scheme, can vary based on refractive index or a change in refractive index of a plasma phase of the initial mixture/first mixture/blood sample, which could be indicative of lipids and/or lipid content.

In one or more exemplary methods, the hematological analysis may be a complete blood cell count. Of course, other analysis may be performed on the blood sample, and this is only one example. In one or more exemplary methods, the complete blood cell count may be performed via an image capture device, e.g. image capturer, image capturing software. Accordingly, it can be advantageous to lyse, e.g. remove, destroy, eliminate, red blood cells. Further, it can be advantageous to stain, e.g. dye, make optically visible, white blood cells and/or platelets. Thus, the white blood cells and/or platelets can be counted from the complete blood cell count hematological analysis. Accordingly, one or more exemplary methods can prepare a blood sample by staining and/or lysing the blood sample based on parameter(s), such as the first blood parameter, of the blood sample. The preparing can be adjusted via different combination schemes based on the parameter(s) including the first blood parameter of the initial mixture/first mixture/blood sample.

For example, if a blood sample has a decreased or increased amount of red blood cells over a “standard” amount of red blood cells, it may be advantageous to add more of a lysing reagent to the mixture, such as the initial mixture and/or the first mixture, or add a stronger lysing reagent to remove the red blood cells. Low concentration of red blood cells can dilute the reagent and reduce lysing activity, and high concentration of red blood cells can require more and/or less reagent to lyse. This can be determined before any hematological analysis is performed, which can greatly speed up the process and eliminate faulty blood samples/test results. By properly preparing the mixture prior to any hematological analysis, a faster and more accurate analysis can be performed.

In one or more exemplary methods, if a blood sample has an increased or decreased amount of red blood cells over a “standard” amount of red blood cells, it may be advantageous to perform mixing and/or or incubation according to a combination scheme, such as the first combination and/or a second combination scheme, to completely remove the red blood cells. This can be determined before any hematological analysis is performed, which can greatly speed up the process and eliminate faulty blood samples. By properly preparing the mixture prior to any hematological analysis, a faster and more accurate analysis can be performed.

As another example, if a blood sample has an increased amount of lipids and/or cholesterol over a “standard” amount of lipids and/or cholesterol, it may be advantageous to add more of a lysing reagent to the initial to remove the red blood cells. This can be determined before any hematological analysis is performed, which can greatly speed up the process and eliminate faulty blood samples. By properly preparing the blood sample prior to any hematological analysis, a faster and more accurate analysis can be performed.

In one or more exemplary methods, if a blood sample has an increased amount of lipids and/or cholesterol over a “standard” amount of lipids and/or cholesterol, it may be advantageous to perform mixing and/or or incubation according to a combination scheme, such as the first combination and/or a second combination scheme, to completely remove the red blood cells. This can be determined before any hematological analysis is performed, which can greatly speed up the process and eliminate faulty blood samples. By properly preparing the mixture prior to any hematological analysis, a faster and more accurate analysis can be performed.

The preparation can occur in a device, e.g. module, machine, housing, system, module, analyzer separate from the hematological analysis device, e.g. module, machine, housing, system, module, analyzer. For example, the preparing device may be physically separate from the hematological analysis device (e.g., physical separated, not physically connected, close by). Alternatively, the preparation can occur in a device, e.g. module, machine, housing, system, module, analyzer combined with the hematological analysis device, e.g. module, machine, housing, system, module, analyzer.

In one or more exemplary methods, the method is performed by a blood analyzer or hematological analysis device. Thus, once a blood sample is inserted into the device, the preparation, combining, monitoring, and hematological analysis can all occur without any user touching the blood sample again. Alternatively, a user can move a prepared mixture to the hematological analysis device. In this instance, the preparation device can be separate, e.g., may not be in fluid communication, from the hematological analysis device.

In one or more exemplary methods, a blood sample, e.g. blood, can be received, e.g. obtained, provided. The blood sample can be from a patient. For example, a blood sample can be withdrawn from a patient. Alternatively, the blood sample may already have been withdrawn from the patient prior to the hematology. The patient may be directly connected to the device.

The blood sample, such as the aspirated blood sample, may be 5, 10, 15, 20, 25, 30, 35, 40, 45, or 50 mL. The blood sample, such as the aspirated blood sample, may be greater than 5, 10, 15, 20, 25, 30, 35, 40, 45, or 50 mL. The blood sample, such as the aspirated blood sample, may be less than 5, 10, 15, 20, 25, 30, 35, 40, 45, or 50 mL.

The blood sample, such as the aspirated blood sample, may be 5, 10, 15, 20, 25, 30, 35, 40, 45, 50, 55, 60, 65, or 70 μL. The blood sample, such as the aspirated blood sample, may be greater than 5, 10, 15, 20, 25, 30, 35, 40, 45, 50, 55, 60, 65, or 70 μL. The blood sample, such as the aspirated blood sample, may be less than 5, 10, 15, 20, 25, 30, 35, 40, 45, 50, 55, 60, 65, or 70 μL. The blood sample, such as the aspirated blood sample, may be between 10 μL and 120 μL.

In one or more exemplary methods, the blood sample may be mixed, e.g. combined, pretreated with an anticoagulant, e.g. blood thinner. The anticoagulant may include, but is not limited to EDTA, coumarins, heparins, synthetic pentasaccharides, citrate, Iloprost, MgSO₄ tubes, and their derivatives/alternatives and combinations. The anticoagulant may be added into the blood sample prior to any hematological testing. In one or more exemplary methods, the anticoagulant can be in a container receiving the blood sample from the patient. In alternative methods, the blood sample is not mixed with an anticoagulant. The inclusion of the anticoagulant can be part of the preparation. The anticoagulant may be part of the blood sample itself.

The blood sample with anticoagulant, such as the aspirated blood sample with anticoagulant, may be 5, 10, 15, 20, 25, 30, 35, 40, 45, or 50 mL. The blood sample with anticoagulant, such as the aspirated blood sample with anticoagulant, may be greater than 5, 10, 15, 20, 25, 30, 35, 40, 45, or 50 mL. The blood sample with anticoagulant, such as the aspirated blood sample with anticoagulant, may be less than 5, 10, 15, 20, 25, 30, 35, 40, 45, or 50 mL.

The blood sample with anticoagulant, such as the aspirated blood sample with anticoagulant, may be 5, 10, 15, 20, 25, 30, 35, 40, 45, 50, 55, 60, 65, or 70 μL. The blood sample with anticoagulant, such as the aspirated blood sample with anticoagulant, may be greater than 5, 10, 15, 20, 25, 30, 35, 40, 45, 50, 55, 60, 65, or 70 μL. The blood sample with anticoagulant, such as the aspirated blood sample with anticoagulant, may be less than 5, 10, 15, 20, 25, 30, 35, 40, 45, 50, 55, 60, 65, or 70 μL. The blood sample with anticoagulant, such as the aspirated blood sample with anticoagulant, may be between 10 μL and 120 μL.

In one or more exemplary methods, saline can be added into the blood sample. This can dilute the sample. The dilution may be taken in consideration for one or more of the methods detailed below. In alternative embodiments, no saline is added to the blood sample. Thus, the blood sample may not be diluted with saline.

The blood sample can be received in a container. The container could be a tube. The container could be a capillary tube. The container could be a cylinder. The container could be a vacuum tube. The container could be a cuvette. The container can be a syringe. The container can be sized and configured to fit within an inlet, e.g. inlet module, opening, aperture, gap, slot, receiver in a system, e.g. housing, system, machine, tester, multi-component system, for providing hematological analysis on the blood sample. The container may provide a fluid connection between a blood sample and the inlet. The container can be a multi-use container. The inlet may be in a housing or other system.

Thus, the blood sample can be received by the device and/or system to perform one or more exemplary methods. Once the blood sample is received, it may follow one or more fluid pathways. These fluid pathways can be, for example, tubes, pipes, capillaries, conduits, hoses, ducts, etc. The method steps discussed herein may all occur on the fluid pathway.

In one or more exemplary methods, the fluid pathway may end at a waste module. The blood sample may be disposed in the waste module.

Once received by the system, the method can be a method of preparation of a blood sample with a reagent for hematology.

In one or more exemplary methods, the method can include combining a blood sample with a reagent. For example, a blood sample can be combined with an initial volume of a first reagent. The combining can provide for provision of an initial mixture. Thus, the initial mixture can be a combination of the blood sample with the initial volume of the first reagent. In one or more exemplary methods, the combining can include mixing the blood sample with the initial volume of the first reagent. This can be done for provision of the initial mixture. The mixing can be performed as discussed in detail below. A liquid sensor may indicate when the initial volume of the first reagent can be combined with the blood sample.

In one or more exemplary methods, an initial volume of the first reagent can be in the range from 0.5 to 20 times a volume of the blood sample in the initial mixture. The initial volume can be 0.5, 1, 2, 3, 4, 5, 6, 7, 8, 9, 10, 11, 12, 13, 14, 15, 16, 17, 18, 19, or 20 times the volume of the blood sample in the initial mixture. The initial volume can be less than 0.5, 1, 2, 3, 4, 5, 6, 7, 8, 9, 10, 11, 12, 13, 14, 15, 16, 17, 18, 19, or 20 times the volume of the blood sample in the initial mixture. The initial volume can be greater than 0.5, 1, 2, 3, 4, 5, 6, 7, 8, 9, 10, 11, 12, 13, 14, 15, 16, 17, 18, 19, or 20 times the volume of the blood sample in the initial mixture.

In one or more exemplary methods, a blood sample to reagent ratio can be 1:1, 1:2, 1:3, 1:4, or 1:5. Accordingly, 20 μL of a blood sample can have 20, 30, 40, 50, 60, 70, 80, 90, or 100 μL of reagent added. In one or more exemplary methods, the blood sample to reagent ratio can be greater than 1:1, 1:2, 1:3, 1:4, or 1:5. In one or more exemplary methods, the blood sample to reagent ratio can be less than 1:1, 1:2, 1:3, 1:4, or 1:5. In particular, the ratio of blood sample to reagent can be significantly below 1:10000, 1:200, 1:100, and 1:50 as typically required in laboratory settings.

The initial volume of the first reagent may be 5, 10, 15, 20, 25, 30, 35, 40, 45, 50, 60, 70, 80, 90, 100, 110, or 120 μL. The initial volume of the first reagent may be greater than 5, 10, 15, 20, 25, 30, 35, 40, 45, 50, 60, 70, 80, 90, 100, 110, or 120 μL. The initial volume of the first reagent may be less than 5, 10, 15, 20, 25, 30, 35, 40, 45, 50, 60, 70, 80, 90, 100, 110, or 120 μL.

Once the blood sample and the initial volume of the first reagent are combined to form the initial mixture, the initial mixture may proceed via an initial combination scheme. Combination schemes are discussed in detail below. The initial combination scheme may include a mixing setting and/or an incubation setting, also discussed below. Thus, the initial mixture may be mixed and/or incubated. The initial combination scheme may not include a volume setting. Alternatively, the initial combination scheme may include a volume setting indicative of the initial volume of the first reagent discussed above.

The initial combination scheme may not be based on any blood sample parameters, such as the first blood parameter as discussed herein. Thus, the initial combination scheme may include a “standard” mixing setting and/or “standard” incubation setting. Alternatively, the initial combination scheme may be based on previous blood parameters received. Thus, the previous blood parameters may be obtained prior to the first blood parameter as discussed herein. The previous blood parameters may have been obtained prior to the combining the blood sample with the first volume of first reagent. Alternatively, the initial combination scheme may be based on standard settings provided by a user.

In one or more exemplary methods, the method can include monitoring the initial mixture, also denoted initial solution. The monitoring the initial mixture may occur after the initial combination scheme is performed. The monitoring the initial mixture may occur before the initial combination scheme is performed. In one or more exemplary methods, the monitoring can include obtaining a first blood parameter. In one or more exemplary methods, the monitoring the initial mixture can include obtaining a first blood parameter of a first blood component of the initial solution. The monitoring can be performed continuously. The monitoring can be performed periodically. The monitoring can be performed at specific points along the method, such as along a fluid pathway that the initial mixture passes through.

In one or more exemplary methods, the monitoring the initial mixture can include monitoring via image capturing and/or via an optical diffraction tomography and/or via a turbidity sensor and/or via a modified liquid sensor and/or via a porous mirror and/or via a spectrophotometer and/or via an electro/impedance measuring unit.

Accordingly, the monitoring can be performed via one or more sensors and/or devices. The monitoring can be performed via a plurality of sensors and/or devices. Via the monitoring, certain parameters, e.g., data, can be obtained regarding the initial mixture and/or the blood sample.

In one or more exemplary methods, the obtaining the first blood parameter can occur during the monitoring. For example, monitoring the initial mixture can comprise obtaining a first blood parameter. The monitoring the initial mixture can include monitoring via image capturing. The monitoring the initial mixture can include monitoring via an optical diffraction tomography. The monitoring the initial mixture can include monitoring via a turbidity sensor. The monitoring the initial mixture can include monitoring via modified liquid sensor. The monitoring the initial mixture can include monitoring via a porous mirror. The monitoring the initial mixture can include monitoring via a spectrograph. The monitoring the initial mixture can include monitoring via an electro/impedance measuring unit.

In one or more exemplary methods, a first blood parameter can be obtained from the initial mixture. Specifically, the first blood parameter can be obtained from a first blood component of the initial mixture. Thus, in one or more exemplary methods the first blood parameter can be from a first blood component, and not from the initial mixture as a whole. For example, this may not include a volume of the mixture. Though in alternate embodiments this may include the volume of the mixture.

The first blood parameter may be any number of parameters of the first blood component. In one or more exemplary methods, the first blood parameter may be a red blood cell content. In one or more exemplary methods, the first blood parameter may be a plasma content. In one or more exemplary methods, the first blood parameter may be a lipid content. For example, the lipid content may be from a sample quality index, such as a HIL index. In one or more exemplary methods, the first blood parameter may be a white blood cell content. In one or more exemplary methods, the first blood parameter may be a blood gas content. In one or more exemplary methods, the first blood parameter may be a platelet content. In one or more exemplary methods, the first blood parameter may be a protein content. In one or more exemplary methods, the first blood parameter may be a cholesterol content. In one or more exemplary methods, the first blood parameter may be total cholesterol content. In one or more exemplary methods, the first blood parameter may be LDL content. In one or more exemplary methods, the first blood parameter may be HDL content. In one or more exemplary methods, the first blood parameter may be a ratio between LDL and HDL. In one or more exemplary methods, the first blood parameter may be triglyceride content. In one or more exemplary methods, the first blood parameter may be a hematocrit content. The first blood parameter may be more than one of the parameters discussed herein, e.g. a combination of two or more. Other first blood parameters can be used as well, and the particular blood parameter is not limiting. In one or more exemplary methods, the first blood parameter can be obtained during aspiration of the blood sample. The first blood parameter can be obtained after aspiration of the blood sample.

In one or more exemplary methods, the first blood parameter may be a red blood cell content indicative of certain contents of the first blood component. For example, the red blood cell content may be indicative of a plasma content of the mixture. The red blood cell content may be indicative of a lipid content of the blood sample. In one or more exemplary methods, the first blood parameter may be a glucose content. In one or more exemplary methods, the first blood parameter may be an electrolyte content. The red blood cell content may be indicative of both a lipid content and a plasma content of the blood sample.

In one or more exemplary methods, the first blood parameter is indicative of a red blood cell content in the initial mixture/blood sample. In one or more exemplary methods, the first blood parameter can be indicative of a white blood cell content in the initial mixture/blood sample. In one or more exemplary methods, the first blood parameter can be indicative of a platelet content in the initial mixture/blood sample. In one or more exemplary methods, the first blood parameter can be indicative of a lipid content in the initial mixture/blood sample. In one or more exemplary methods, the first blood parameter is indicative of a red blood cell content in the initial mixture and/or a white blood cell content in the initial mixture and/or a platelet content in the initial mixture and/or a lipid content in the initial mixture.

The obtaining may be performed via a number of different methods. In one or more exemplary methods, the obtaining the first blood parameter of the first blood component of the initial mixture comprises obtaining image data of the mixture. In one or more exemplary methods, the obtaining the first blood parameter of the first blood component of the initial mixture comprises obtaining sensor data of the mixture. In one or more exemplary methods, the obtaining the first blood parameter of the first blood component of the initial mixture comprises obtaining image data and/or sensor data of the mixture.

In one or more exemplary methods, the first blood parameter may be obtained directly. In one or more exemplary methods, the first blood parameter may be obtained indirectly. For example, a sum of electrolytes and similar analytes of the osmolality may be measured, which can affect the ability to lyse and stain cells.

The first blood parameter can be determined from the image data. The first blood parameter can be determined from the sensor data. The first blood parameter can be determined from the image data and/or the sensor data.

Monitoring via image data may be performed via taking photographs, e.g. images. For example, the images can be taken from one or more cameras. Alternatively, other imaging systems, e.g., image capturing systems, image capturer can perform the obtaining the image data. The image capturing system may be associated with a fluid pathway of the mixture. The image capturing system may output data indicative of the first blood parameter.

Obtaining the sensor data may be performed via one or more sensors, e.g. detectors, electronics. The sensor(s) may be along the fluid pathway in order to obtain the sensor data while the blood sample passes through the system. The sensors may output data indicative of the first blood parameter.

In one or more exemplary methods, the sensor may be an absorbance sensor and/or a transmittance sensor and/or a refractive index sensor and/or a liquid sensor and/or a hematocrit sensor.

As mentioned, the sensor may be an impedance sensor. The impedance sensor may provide sensor data, which may be indicative of the first blood parameter. The impedance sensor may provide a first blood parameter indicative of electrolyte content. The impedance sensor may provide a first blood parameter indicative of lipid content. The impedance sensor may provide a first blood parameter indicative of hematocrit content. The impedance sensor may provide a first blood parameter indicative of electrolyte content and/or lipid content and/or hematocrit content.

In one or more exemplary methods, the obtaining the first blood parameter comprises monitoring via a blood gas (BG) analysis. The blood gas analysis may include one or more of blood gas, e.g. pH, pCO₂, pO₂, electrolytes, metabolites, and oximetry (CO—OX).

In one or more exemplary methods, the obtaining the first blood parameter can include hemolysis detection. The hemolysis detection can be sensitive to refraction index changes from lipids.

In one or more exemplary methods, the obtaining the first blood parameter comprises sensing one or more electrical and/or optical properties of the initial mixture. In other words, the first blood parameter, and optionally further blood parameters may be indicative of an electrical and/or optical property of the initial mixture.

In one or more exemplary methods, the obtaining the first blood parameter comprises performing a conductivity analysis of the initial mixture. In one or more exemplary methods, the obtaining the first blood parameter comprises performing a conductivity analysis. The conductivity analysis can determine the hematocrit of the initial mixture, e.g. the volume percentage of red blood cells. For example, electrodes can be used to measure the conductivity. Osmolality can be measured/determined/estimated from the conductivity. The electrodes can span the fluid pathway in order to obtain sensor data of the initial mixture/first mixture/second mixture. The electrodes can be incorporated into the fluid pathway.

In one or more exemplary methods, the obtaining the first blood parameter comprises performing a light intensity analysis of the initial mixture. In one or more exemplary methods, the obtaining the first blood parameter comprises performing a light intensity analysis e.g. at one or more different wavelengths. For example, an optical sensor can be used. The optical sensor and/or light source can be at 509, 522, 549, 569, 587, 650, 660, 670, 680, or 690 nm.

In one or more exemplary methods, the obtaining the first blood parameter comprises performing a viscosity analysis of the initial mixture. In one or more exemplary methods, the obtaining the first blood parameter comprises performing a viscosity analysis.

In one or more exemplary methods, the obtaining the first blood parameter comprises performing a pH analysis of the initial mixture. In one or more exemplary methods, the obtaining the first blood parameter comprises performing a pH analysis.

In one or more exemplary methods, the obtaining the first blood parameter comprises using a hemolysis detector to analyze a plasma phase of the blood sample, e.g. for detecting lipids or lipid levels in the blood sample.

In one or more exemplary methods, multiple sensors, such as optical sensors, and/or image capturers may be used. Thus, multiple different parameters can be obtained from the initial mixture after preparing the blood sample with the initial volume of reagent. Alternatively, multiple sensors and/or image capturers may be used to obtain and verify the same blood parameter.

The obtaining either the sensor data and/or the image data may be performed via a controller. For example, a user may activate the system to obtain the data. Alternatively, the obtaining either the sensor data and/or the image data may be performed automatically e.g. as a part of a full CBC cycle. A computer system may determine a particular time, such as an optimal time, to obtain the first blood parameter. Thus, the computer system may be in electronic communication with the sensors and/or image capturer. The computer system may further include a processor to process the obtained sensor and/or image data. The computer system may be able to output the processed data.

After obtaining the first blood parameter, one or more exemplary methods can include determining a first combination scheme. Unlike the initial combination scheme, the first combination scheme can be based on the first blood parameter. The first combination scheme can be one or more combination schemes. The first combination scheme can be selected, e.g. determined, from a list, e.g. group, table, of different combination schemes. For example, the first combination scheme can be selected from the list based on the particular first blood parameter.

In one or more exemplary methods, the list can include a plurality, 3, 4, 5, 10, 15, 20, 25, 30, 35, 40, 50, 60, 70, 80, 90, or 100 different combination schemes. In one or more exemplary methods, the list can include greater than 5, 10, 15, 20, 25, 30, 35, 40, 50, 60, 70, 80, 90, or 100 different combination schemes. In one or more exemplary methods, the list can include less than 5, 10, 15, 20, 25, 30, 35, 40, 50, 60, 70, 80, 90, or 100 different combination schemes.

In one or more exemplary methods, the first combination scheme can vary depending on the first blood parameters. Thus, different combination schemes may be relevant to different first blood sample parameters. For example, if the initial mixture has a high lipid level, the combination scheme relevant to an initial mixture with high lipid content can be selected as the first combination scheme. Another combination scheme may be relevant to initial mixtures with high or low red blood cell content and/or high platelet content and can be selected as the first combination scheme, e.g. if the first blood parameter is indicative of high red blood cell content and/or high platelet content. Another combination scheme may be relevant to low red blood cell content and high protein content and can be selected as the first combination scheme, e.g. if the first blood parameter is indicative of low red blood cell content and high protein content. Other iterations of combination schemes can be used as well. This can allow for different combination schemes to be used depending on the first blood parameter, providing for more accurate results.

Further, the method can include preparing the initial mixture/blood sample according to the first combination scheme. Different combination schemes can include different combination settings as discussed below, which may be relevant to the first blood parameter. Thus, advantageously, one or more of the exemplary methods can focus the combination settings of the first combination scheme to properly adjust the combination settings for the particular parameter(s) of the blood sample. This can lead to accurate and fast hematological results.

For example, the method can include preparing the initial mixture with a reagent according to the first combination scheme, e.g. by adding a first volume of the first reagent and/or a first volume of a second reagent to the initial mixture according to the first combination scheme. In other words, determining a combination scheme, such as the first combination scheme and/or second combination scheme, may comprise selecting one or more reagents from a set of reagents for the combination scheme.

The reagent can be one or more reagents that can be mixed with, e.g. incorporated into, combined with, added into, the initial mixture. The reagent can be configured to affect one or more properties of the initial mixture. For example, the reagent can be a staining reagent, e.g. stain the initial mixture. This can make certain components of the blood, such as white blood cells and platelets, optically visible for analysis. The reagent can be a lysing reagent, e.g. to lyse the initial mixture. The lysing can affect the red blood cells. A single reagent can be a lysing reagent and a staining reagent. Multiple reagents could be used as well, with one reagent being a lysing reagent and another being a staining reagent.

Thus, as discussed above an initial volume of a first reagent is combined with the blood sample to form an initial mixture. In other words, the blood sample may be combined or prepared with first reagent according to an initial combination scheme defining the initial volume. However, the initial volume of a first reagent may not have sufficiently lysed or stained the blood sample. Further, the initial combination scheme may not be tailored to any particular blood parameters. This could lead to inaccurate hematological results. Accordingly, it can be advantageous to add more reagent into the initial mixture and/or incubate the initial mixture and/or mix the initial mixture. Thus, the blood sample in the initial mixture may go through further preparing, such as staining and/or lysing, leading to more accurate results.

Thus, advantageously, a user could add a lesser amount of reagent in the initial volume and monitor the initial mixture. Based on the first blood parameter obtained during the monitoring, additional and/or different reagent can be adjusted based on a first combination scheme to best lyse and/or stain the initial mixture. This can allow for more precise usage of the reagent, leading to improved hematological results.

In one or more exemplary methods, the method can include adding a first volume of the first reagent to the initial mixture, e.g., adjusting a reagent volume. This can be performed according to the first combination scheme. Thus, the adding can be performed for provision of a first mixture.

The first volume of first reagent may be greater than the initial volume of first reagent. The first volume of first reagent may be less than the initial volume of first reagent. The first volume of first reagent may be the same as the initial volume of first reagent.

The first volume of the first reagent may be 5, 10, 15, 20, 25, 30, 35, 40, 45, 50, 60, 70, 80, 90, 100, 110, or 120 μL. The first volume of the first reagent may be greater than 5, 10, 15, 20, 25, 30, 35, 40, 45, 50, 60, 70, 80, 90, 100, 110, or 120 μL. The first volume of the first reagent may be less than 5, 10, 15, 20, 25, 30, 35, 40, 45, 50, 60, 70, 80, 90, 100, 110, or 120 μL.

In one or more exemplary methods, the method can include adding a first volume of a second reagent to the initial mixture, e.g. adjusting reagent levels. This can be performed according to the first combination scheme. Thus, the adding can be performed for provision of a first mixture.

A second reagent may be used for a number of reasons. For example, the first reagent may be a lysing reagent, and the second reagent may be a staining reagent. Alternatively, the first reagent may be a staining reagent, and the second reagent may be a lysing reagent. In one or more exemplary methods, the first reagent can be a staining and/or lysing reagent. In one or more exemplary methods, the second reagent is a staining and/or lysing reagent. In or more exemplary methods, the first reagent and/or the second reagent may be a dilution agent. For example, the first reagent and/or the second reagent may be saline.

In one or more exemplary methods, the first reagent may be a lysing and staining reagent, and the second reagent may be a lysing and staining reagent. The second reagent may be a weaker lysing and staining reagent than the first reagent. The second reagent may be a stronger lysing and staining reagent than the first reagent.

The second reagent may have a stronger lysing component and the same staining component as the first reagent. The second reagent may have a weaker lysing component and the same staining component as the first reagent. The second reagent may have a stronger lysing component and a weaker staining component as the first reagent. The second reagent may have a weaker lysing component and a stronger staining component as the first reagent.

The first volume of second reagent may be greater than the initial volume of first reagent. The first volume of second reagent may be less than the initial volume of first reagent. The first volume of second reagent may be the same as the initial volume of first reagent.

The first volume of second reagent may be greater than the first volume of first reagent. The first volume of second reagent may be less than the first volume of first reagent. The first volume of second reagent may be the same as the first volume of first reagent.

The first volume of the second reagent may be 5, 10, 15, 20, 25, 30, 35, 40, 45, 50, 60, 70, 80, 90, 100, 110, or 120 μL. The first volume of the second reagent may be greater than 5, 10, 15, 20, 25, 30, 35, 40, 45, 50, 60, 70, 80, 90, 100, 110, or 120 μL. The first volume of the second reagent may be less than 5, 10, 15, 20, 25, 30, 35, 40, 45, 50, 60, 70, 80, 90, 100, 110, or 120 μL.

In one or more exemplary methods, the first reagent could be added to the initial mixture/blood sample along a fluid pathway from the inlet. For example, the system can include a first reagent inlet in fluid communication with the pathway. This can be at a T-junction. The first reagent inlet can be in fluid communication with a first reservoir, e.g. housing, container, storage, for accommodating the first reagent. Thus, the first reagent can be in fluid communication with the fluid pathway that the initial mixture/blood sample is held within. In one or more exemplary methods, the first reagent inlet can include a dose valve for proper addition of reagent, such as first reagent, to the initial mixture/blood sample. The valve can be controlled by a user, or electronically such as through a computer system or controller as disclosed herein.

In one or more exemplary methods, the second reagent could be added to the initial mixture/blood sample along a fluid pathway from the inlet. For example, the system can include a second reagent inlet in fluid communication with the pathway. This can be at a T-junction. The second reagent inlet can be in fluid communication with a second reservoir, e.g., housing, container, storage, for accommodating the second reagent. Thus, the second reagent can be in fluid communication with the fluid pathway that the initial mixture/blood sample is held within. In one or more exemplary methods, the second reagent inlet can include a dose valve for proper addition of reagent to the blood sample. The valve can be controlled by a user, or electronically such as through a computer system or controller as disclosed herein.

As will be discussed further in detail below, the initial mixture and/or first mixture may undergo mixing. In one or more exemplary methods, the initial mixture and/or first mixture may undergo incubating. In one or more exemplary methods, the initial mixture and/or first mixture may undergo both mixing and incubating. As will be discussed, settings of the mixing and incubating can be included in the combination schemes, such as the initial combination scheme and/or the first combination scheme and/or the second combination scheme.

In one or more exemplary methods, the method can further include mixing of the initial mixture and/or first mixture. For example, reagent can be mixed with the blood sample after forming the initial mixture. While adding the reagent to the fluid pathway with the initial mixture/blood sample some mixing may be performed, and it may be advantageous to further mix the initial mixture/blood sample and the reagent to form a first mixture.

Thus, the system can include a mixer. The mixer may be in fluid communication with or form a part of the fluid pathway. Thus, the initial mixture can pass through the mixer. The mixer can be a helical mixer. For example, the mixer can use a helix principle. The mixer can be a centrifuging mixer and/or a static mixer and/or a high shear mixer and/or a drum mixer. Preferably, the mixer does not activate any components of the blood sample, such as causing consolidation of platelets and thereby causing clotting. The mixer can provide a shear force onto the initial mixture. Increasing certain settings of the mixer, such as discussed below, can increase the likelihood of red blood cell lysis, thus providing for improved hematological analysis. For example, by increasing mixing cycles or mixing speeds, shear forces on the cells are increased, thereby improving red blood cell lysis.

By passing through the mixer, the mixer may mix the reagent with the blood sample of the initial mixture and any subsequent mixtures. The initial mixture/first mixture may pass through the mixer one or more times, such as back and forth. For example, the initial mixture/first mixture may pass through the mixer in a first direction. The mixture then may reverse to a second direction and pass through the mixer again, thereby further mixing the initial mixture/first mixture. The initial mixture or first mixture may then reverse to the first direction to pass through the mixer a third time, such as according to the initial combination scheme or first combination scheme. Each pass through the mixer may be known as a cycle.

In one or more exemplary methods, the method can further include incubating, e.g. heating the initial mixture and/or first mixture. The incubator may be configured to heat a portion of the fluid pathway, thus heating the fluid pathway. The incubator may be located after the mixer. The incubator may be located prior to the mixer.

In one or more exemplary methods, the mixer may also be an incubator. The incubator can be a component of the mixer. Thus, the mixer can be configured to incubate and mix the initial mixture and/or first mixture.

In one or more exemplary embodiments, the monitoring can occur after the mixer. In one or more exemplary embodiments, the monitoring can occur after the incubator. In one or more exemplary embodiments, the monitoring can occur after the combination mixer-incubator.

In one or more exemplary embodiments, the monitoring can occur in the mixer. In one or more exemplary embodiments, the monitoring can occur in the incubator. In one or more exemplary embodiments, the monitoring can occur in the combination mixer-incubator.

In one or more exemplary embodiments, the monitoring can occur before the mixer. In one or more exemplary embodiments, the monitoring can occur before the incubator. In one or more exemplary embodiments, the monitoring can occur before the combination mixer-incubator.

In one or more exemplary embodiments, the adding a first volume of a first reagent and/or a first volume of a second agent may occur in front of the mixer (before mixing and/or incubation in the mixer), e.g. closer to the inlet. In one or more exemplary embodiments, the adding a first volume of a first reagent and/or a first volume of a second agent may occur in the mixer. In one or more exemplary embodiments, the adding a first volume of a first reagent and/or a first volume of a second agent may occur behind the mixer (after mixing and/or incubation in the mixer), e.g. further from the inlet.

In one or more exemplary embodiments, the adding a first volume of a first reagent and/or a first volume of a second agent may occur in front of the incubator, e.g. closer to the inlet. In one or more exemplary embodiments, the adding a first volume of a first reagent and/or a first volume of a second agent may occur in the incubator. In one or more exemplary embodiments, the adding a first volume of a first reagent and/or a first volume of a second agent may occur behind the incubator, e.g. further from the inlet.

In one or more exemplary embodiments, the adding a first volume of a first reagent and/or a first volume of a second agent may occur in front of the combination mixer-incubator, e.g. closer to the inlet. In one or more exemplary embodiments, the adding a first volume of a first reagent and/or a first volume of a second agent may occur in the combination mixer-incubator. In one or more exemplary embodiments, the adding a first volume of a first reagent and/or a first volume of a second agent may occur behind the combination mixer-incubator, e.g. further from the inlet.

As mentioned above, the initial mixture may be prepared according to an initial combination scheme, which can include any or all of the settings discussed below. The initial combination scheme may or may not be based on any blood parameters.

Further, a first combination scheme and/or second combination scheme may be determined based on the first blood parameter. The combination scheme, such as the initial combination scheme and/or the first combination scheme and/or the second combination scheme, may include one or more settings. As discussed below, the combination scheme, such as the initial combination scheme and/or the first combination scheme and/or the second combination scheme, may include a volume setting and/or a mixing setting and/or an incubation setting. The combination scheme, such as the initial combination scheme and/or the first combination scheme and/or the second combination scheme, may include a volume setting and a mixing setting. The combination scheme, such as the initial combination scheme and/or the first combination scheme and/or the second combination scheme, may include a volume setting and an incubation setting. The combination scheme, such as the initial combination scheme and/or the first combination scheme and/or the second combination scheme, may include a mixing setting and an incubation setting. The combination scheme can include any number of settings for different modules and/or components and/or methodologies as would be advantageous in preparing a blood sample for hematological analysis.

In one or more exemplary methods, different combination schemes may include different settings. For example, the initial combination scheme may include a mixing setting and an incubation setting whereas the first combination scheme may include a mixing setting, an incubation setting, and a volume setting. The particular settings incorporated into each combination scheme is not limiting.

In one or more exemplary methods, the combination scheme, such as the initial combination scheme and/or the first combination scheme and/or the second combination scheme, may include a volume setting for adding an initial volume and/or a first volume of a first reagent and/or a second reagent, such as a first volume setting and/or a second volume setting and/or a third volume setting, etc., as discussed in detail above. Thus, the combination scheme, such as the initial combination scheme and/or the first combination scheme and/or the second combination scheme, can include a first volume setting. In one or more exemplary methods, the first volume setting can be indicative of a first volume of the reagent. In one or more exemplary methods, the first volume setting can be indicative of a total volume of the reagent. In one or more exemplary methods, the first volume setting can be indicative of a first volume of the reagent as compared to a volume of the blood sample, such as a first volume ratio. In one or more exemplary methods, the first volume setting can be indicative of a first volume of the reagent and includes adding a first volume of the reagent to the blood sample in a volume according to the first volume setting.

In one or more exemplary methods, the combination scheme, such as the initial combination scheme and/or the first combination scheme and/or the second combination scheme, may include a mixing setting, such as an initial mixing settings and/or a first mixing setting and/or a second mixing setting and/or a third mixing setting, etc. Thus, the initial combination scheme can include an initial mixing setting. The first combination scheme can include a first mixing setting. In one or more exemplary methods, the initial mixing setting and/or the first mixing setting can be indicative of a mixing configuration.

For example, the combination scheme, such as the initial combination scheme and/or the first combination scheme and/or the second combination scheme, can be indicative of a mixing configuration for a mixing of the initial mixture. Thus, preparing the blood sample according to the first combination scheme includes mixing the blood sample and the reagent according to the first mixing setting. In one or more exemplary methods, preparing the initial mixture according to the initial combination scheme includes mixing the initial mixture according to the initial mixing setting. In one or more exemplary methods, preparing the initial mixture according to the first combination scheme includes mixing the initial mixture according to the first mixing setting.

A mixing setting, e.g. of a combination scheme, can include one or more parameters of a mixer. For example, a mixing setting can include number of mixing cycles, e.g. times that the mixture/reagent and blood sample pass through the mixer. In one or more exemplary methods, the mixing setting can be 1, 2, 3, 4, 5, 6, 7, 8, 9, or 10 cycles. In one or more exemplary methods, the mixing setting can be less than 2, 3, 4, 5, 6, 7, 8, 9, or 10 cycles. In one or more exemplary methods, the mixing setting can be greater than 1, 2, 3, 4, 5, 6, 7, 8, 9, or 10 cycles.

A mixing setting, e.g. of a combination scheme, can include a flow velocity of the mixture/reagent and blood sample through the mixer. In one or more exemplary methods, the flow velocity can be 5, 10, 15, 20, 25, 30, 35, 40, 45, 50, 55, 60, 65, 70, 75, 80, 85, 90, 95, or 100 L/second. In one or more exemplary methods, the flow velocity can be greater than 5, 10, 15, 20, 25, 30, 35, 40, 45, 50, 55, 60, 65, 70, 75, 80, 85, 90, 95, or 100 L/second. In one or more exemplary methods, the flow velocity can be less than 5, 10, 15, 20, 25, 30, 35, 40, 45, 50, 55, 60, 65, 70, 75, 80, 85, 90, 95, or 100 L/second.

A mixing setting, e.g. of a combination scheme, can include a mixing time in the mixer. In one or more exemplary methods, the mixing time can be 5, 10, 15, 20, 25, 30, 35, or 50 seconds. In one or more exemplary methods, the mixing time can be greater than 5, 10, 15, 20, 25, 30, 35, or 50 seconds. In one or more exemplary methods, the mixing time can be less than 5, 10, 15, 20, 25, 30, 35, or 50 seconds.

A mixing setting, e.g. of a combination scheme, can include one of the mentioned mixing settings, among others. In alternatives, the mixing setting can include two or all of the mentioned mixing settings, among others.

In one or more exemplary methods, the combination scheme, such as the initial combination scheme and/or the first combination scheme and/or the second combination scheme, may include an incubation setting, such as an initial incubation setting and/or a first incubation setting and/or a second incubation setting and/or a third incubation setting, etc. Thus, the initial combination scheme can include an initial incubation setting. The first combination scheme can include a first incubation setting. In one or more exemplary methods, the initial incubation scheme and/or the first incubation setting can be indicative of an incubation configuration. For example, it can be indicative of an incubation configuration, such as the initial combination scheme and/or the first combination scheme and/or the second combination scheme, for the initial mixture. Thus, preparing the blood sample according to the first combination scheme includes incubating the initial mixture/blood sample according to the first incubation setting. In one or more exemplary methods, preparing the initial mixture according to the initial combination scheme includes incubating the initial mixture according to the initial incubation setting. In one or more exemplary methods, preparing the initial mixture according to the first combination scheme includes incubating the initial mixture according to the first incubation setting.

An incubation setting can include one or more parameters of an incubator. For example, this can include an incubation temperature. In one or more exemplary methods, the initial mixture may be incubated at a temperature of 30, 40, 45, 46, 47, 48, 49, 50, or 55° C. In one or more exemplary methods, the initial mixture may be incubated at a temperature of greater than 30, 40, 45, 46, 47, 48, 49, 50, or 55° C. In one or more exemplary methods, the initial mixture may be incubated at a temperature of less than 30, 40, 45, 46, 47, 48, 49, 50, or 55° C.

An incubation setting can include an incubation time. In one or more exemplary methods, the initial mixture may be incubated for 10, 11, 12, 13, 14, 15, 16, 17, 18, 19, 20, 21, 22, 23, 24, or 25 seconds. In one or more exemplary methods, the initial mixture may be incubated for greater than 10, 11, 12, 13, 14, 15, 16, 17, 18, 19, 20, 21, 22, 23, 24, or 25 seconds. In one or more exemplary methods, the initial mixture may be incubated for less than 10, 11, 12, 13, 14, 15, 16, 17, 18, 19, 20, 21, 22, 23, 24, or 25 seconds. In one or more exemplary methods, the initial mixture may not be mixing, e.g. may remain stationary, during the incubation. Alternatively, the initial mixture may be moving during incubation. The incubation time may vary based on the incubation temperature.

The incubation setting can include one of an incubation temperature and an incubation time, among others. In alternatives, the incubation setting can include both of an incubation time and an incubation temperature, among others.

Advantageously, in one or more exemplary methods the first blood parameter can be determined after the mixer and/or incubator, and thus after performing the initial combination scheme. Thus, the initial mixture can be mixed prior to determining the first blood parameter. After determining the first blood parameter, the initial mixture can pass back through the mixer and/or incubator according to the initial combination scheme and/or the first combination scheme. The first volume of the first reagent and/or the first volume of the second reagent can then be added into the initial mixture according to the first combination scheme to form the first mixture. The first mixture can then pass again through the mixer and/or incubator again according to the first combination scheme.

In one or more exemplary methods, the first combination scheme and/or the second combination scheme may include a reagent identifier, e.g., first reagent identifier, of the reagent from a set of available reagent identifiers. Thus, in one or more exemplary methods, preparing the first mixture according to the first combination scheme includes selecting the reagent according to the reagent identifier. For example, as discussed above, a first reagent may have stronger lysing properties than a second reagent. Thus, if stronger lysing may be advantageous, the first reagent may be identified or specified in the combination scheme. In one or more exemplary methods, the method can include outputting a first reagent indicator indicative of the first combination scheme.

Advantageously, the first combination scheme can allow for improved hematological analysis. For example, the first combination scheme is configured to achieve full, e.g. sufficient, blood cell lysis and strong white blood cell and/or platelet staining.

Full, e.g. sufficient, blood cell lysis can be achieved when 95, 96, 97, 98, 99, 99.5, 99.6, 99.7, 99.8, 99.9, or 100% of red blood cells are lysed. Full, e.g. sufficient, blood cell lysis can be achieved when less than 95, 96, 97, 98, 99, 99.5, 99.6, 99.7, 99.8, 99.9, or 100% of red blood cells are lysed. Full, e.g. sufficient, blood cell lysis can be achieved when greater than 95, 96, 97, 98, 99, 99.5, 99.6, 99.7, 99.8, or 99.9% of red blood cells are lysed.

Advantageously, the disclosed methods can achieve minimal white blood cell and/or platelet lysing. In one or more exemplary methods, 5, 4, 3, 2, 1, 0.5, 0.4, 0.3, 0.2, 0.1, or 0.0% of white blood cells and/or platelets are lysed. In one or more exemplary methods, less than 5, 4, 3, 2, 1, 0.5, 0.4, 0.3, 0.2, or 0.1% of white blood cells and/or platelets are lysed. In one or more exemplary methods, greater than 5, 4, 3, 2, 1, 0.5, 0.4, 0.3, 0.2, 0.1, or 0.0% of white blood cells and/or platelets are lysed.

Strong white blood cell and/or platelet staining can be achieved when 95, 96, 97, 98, 99, 99.5, 99.6, 99.7, 99.8, 99.9, or 100% of white blood cells and/or platelets are stained. Strong white blood cell and/or platelet staining can be achieved when less than 95, 96, 97, 98, 99, 99.5, 99.6, 99.7, 99.8, 99.9, or 100% of white blood cells and/or platelets are stained. Strong white blood cell and/or platelet staining can be achieved when greater than 95, 96, 97, 98, 99, 99.5, 99.6, 99.7, 99.8, or 99.9% of white blood cells and/or platelets are stained.

Thus, in one or more exemplary methods, the method can include performing a complete blood count (CBC) analysis of the first mixture and/or the second mixture. This can be done after preparing the blood sample, adding the reagent, and incubating the blood sample and/or mixing the blood sample. The analysis can be performed via a cell measurement component for accommodating the initial mixture. The analysis can be performed via artificial intelligence and/or image analysis. The analysis can be performed via a computer system. The analysis can be performed via counting of the white blood cells and/or platelets.

In one or more exemplary methods, the method can further include obtaining a second blood parameter. The second blood parameter can be obtained at the same monitoring as the first blood parameter. Alternatively, the second blood parameter can be obtained at a different monitoring as the first blood parameter. The second blood parameter can be of a second blood component of the blood sample. The second blood parameter can be of the first blood component of the blood sample. The second parameter may be of the blood sample. Thus, the determining the first combination scheme can be based on the first blood parameter and the second blood parameter. Alternatively, the first combination scheme can be based on the first blood parameter or the second blood parameter. Monitoring and obtaining the second blood parameter may include any or all of the discussion regarding monitoring and obtaining the first blood parameter.

In one or more exemplary methods, the first blood parameter and the second blood parameter can be different parameters. For example, the first blood parameter can be indicative of a red blood cell content of the blood sample, e.g. hematocrit. The second blood parameter can be indicative of a lipid content of the first mixture. However, any of the above-discussed blood parameters can be used for each of the first blood parameter and the second blood parameter.

In one or more exemplary methods, the method can further include obtaining a third, fourth, fifth, etc. blood parameter. Therefore, the combination scheme(s), such as the first combination scheme and/or the second combination scheme, may be based on the first, second, third, fourth, fifth, etc. blood parameter. In one or more exemplary embodiments, the different blood parameters may be ranked. Thus, one parameter may be ranked higher than another parameter. If the two parameters require competing combination schemes, the combination scheme associated with the higher ranked parameter may be determined and used for the preparation of the blood sample.

In one or more exemplary methods, the method can include outputting a combination indicator indicative of the first combination scheme. The combination indicator can be output to a computer system in order to make any adjustments to the initial mixture and/or the first mixture as discussed in detail above. Further, the combination indicator can be output to a user who can manually adjust the initial mixture and/or the first mixture, such as according to the above disclosure. The combination indicator can be electronically communicated, such as through a wireless or wired connector. In one or more exemplary methods, the combination indicator can be output to a visual device, such as a monitor and/or screen and/or laptop and/or table and/or phone.

In one or more exemplary methods, the method may optionally include further steps. For example, the method may include obtaining a quality parameter of the initial mixture and/or the first mixture.

The quality parameter may be indicative of one or more properties of the initial mixture and/or the first mixture. The quality parameter may be indicative of red blood cell lysing of the initial mixture and/or the first mixture. The quality parameter may be indicative remaining red blood cells of the initial mixture and/or the first mixture. The quality parameter may be indicative of staining of white blood cells of the initial mixture and/or the first mixture. The quality parameter may be indicative of staining of platelets of the initial mixture and/or the first mixture.

Further, the method can include determining whether the quality parameter satisfies or does not satisfy one or more quality criterion. The quality criterion can be a numerical value of certain threshold parameters of the first mixture. The quality criterion may be one or more values. For example, the quality criterion may be satisfied if the quality parameter is above the quality criterion. The quality criterion may be satisfied if the quality parameter is below the quality criterion. The quality criterion may be satisfied if the quality parameter is between two values of the quality criterion.

In one or more exemplary methods, the quality criterion may include a first lysis threshold. In one or more exemplary methods, the quality criterion may be met when a first lysis threshold of 95, 96, 97, 98, 99, 99.5, 99.6, 99.7, 99.8, 99.9, or 100% of red blood cells being lysed is met. For example, the quality criterion may be met at a first lysis threshold of 99.5% of red blood cells being lysed. If the quality parameter of the initial mixture and/or the first mixture indicates that 99.6% of red blood cells are lysed, the quality criterion would be satisfied. If 99.4% of red blood cells were lysed, the quality criterion would not be satisfied. Thus, in one or more exemplary methods, the quality parameter is indicative of a degree of red blood cell lysis and wherein the quality criterion is satisfied if the quality parameter is larger than a first lysis threshold.

In one or more exemplary methods, the quality criterion may include a second lysis threshold. In one or more exemplary methods, the quality criterion may be met when 5, 4, 3, 2, 1, 0.5, 0.4, 0.3, 0.2, 0.1, or 0.0% of white blood cells and/or platelets are lysed. For example, the quality criterion may be met at a first lysis threshold 0.2% of white blood cells and/or platelets being lysed. If the quality parameter of the initial mixture and/or the first mixture indicates that 0.3% of white blood cells and/or platelets are lysed, the quality criterion would be satisfied. If 0.1% of white blood cells and/or platelets were lysed, the quality criterion would not be satisfied. Thus, in one or more exemplary methods, the quality parameter can be indicative of a degree of white blood cell lysis and/or platelet lysis and wherein the quality criterion is satisfied if the quality parameter is larger than a second lysis threshold.

In one or more exemplary methods, the quality criterion may include a third lysis threshold. The third lysis threshold may be indicative of staining. In one or more exemplary methods, the quality criterion may be met when 90, 91, 92, 93, 94, 95, 96, 97 98, or 99% of white blood cells and/or platelets are stained. The quality criterion may be met when greater than 90, 91, 92, 93, 94, 95, 96, 97 98, or 99% of white blood cells and/or platelets are stained. The quality criterion may be met when less than 90, 91, 92, 93, 94, 95, 96, 97 98, or 99% of white blood cells and/or platelets are stained.

In one or more exemplary methods, the quality criterion may include a fourth lysis threshold. The fourth lysis threshold may be indicative of remaining red blood cells. In one or more exemplary methods, the quality criterion may be met when 5, 4, 3, 2, 1, 0.5, 0.4, 0.3, 0.2, 0.1, or 0.0% of red blood cells remain, e.g. have not been lysed. The quality criterion may be met when greater than 5, 4, 3, 2, 1, 0.5, 0.4, 0.3, 0.2, 0.1, or 0.0% of red blood cells remain, e.g. have not been Iysed. The quality criterion may be met when less than 5, 4, 3, 2, 1, 0.5, 0.4, 0.3, 0.2, 0.1, or 0.0% of red blood cells remain, e.g. have not been lysed.

In accordance with a determination that the quality criterion is satisfied, the initial mixture and/or the first mixture may continue to be analyzed, such as discussed herein. For example, in accordance with a determination that the quality criterion is satisfied, performing a complete blood count (CBC) analysis of the initial mixture and/or the first mixture. In one or more exemplary methods, the method comprises performing a complete blood count (CBC) analysis of the initial mixture and/or the first mixture irrespective of a quality criterion or when a quality criterion is not available. In other words, the quality criterion may be omitted.

In accordance with a determination that the quality criterion is not satisfied, other procedures may be taken. For example, if the quality criterion of the initial mixture is not satisfied, the method may move to determining the first combination scheme. If the quality criterion of the first mixture is not satisfied, the method may include determining a second combination scheme, as mentioned below. The second combination scheme may include any and all of the settings discussed above with regard to the first combination scheme and/or the second combination scheme, such as the mixing setting, the volume setting, and/or the incubation setting. Thus, in one or more exemplary methods, the method may include adding a second volume of the first reagent to the first mixture according to the second combination scheme for provision of a second mixture. The method may include adding a second volume of the second reagent to the first mixture according to the second combination scheme for provision of a second mixture. The first mixture may undergo additional mixing and/or incubation according to the second combination scheme.

Thus, advantageously the method can be repeated until the quality criterion is met. For example, a second mixture, third mixture, fourth mixture, etc. can be formed and may undergo a respective second combination scheme, third combination scheme, fourth combination scheme, etc. Thus, it will be understood that the second, third, fourth, etc. mixture may include any or all of the discussions relevant to the first mixture. Further, it will be understood that the second, third, fourth, etc. combination schemes may include any or all of the discussions relevant to the first combination scheme.

Thus, one or more exemplary methods may be iterative. Reagent can be added in small doses to advantageously properly lyse the red blood cells and/or stain the white blood cells and/or platelets without lysing the white blood cells and/or platelets.

In one or more exemplary embodiments, no additional reagent may be added to the initial mixture. In one or more exemplary embodiments, one additional volume of reagent is added. In one or more exemplary embodiments, 2, 3, 4, 5, 6, 7, 8, 9, or 10 additional volumes of reagent are added. In one or more exemplary embodiments, less than 1, 2, 3, 4, 5, 6, 7, 8, 9, or 10 additional volumes of reagent are added. In one or more exemplary embodiments, greater than 1, 2, 3, 4, 5, 6, 7, 8, 9, or 10 additional volumes of reagent are added.

As mentioned, the initial mixture and/or the first mixture can be analyzed. In one or more exemplary methods, the initial mixture and/or the first mixture can be introduced to a container (e.g., cuvette), which can then be analyzed. For example, the initial mixture and/or the first mixture can be image analyzed and/or sensor analyzed. The container can be a multi-use container. Alternatively, the container can be single use.

Advantageously, one or more exemplary methods can provide advantages for hematological analysis. For example, the overall preparation time before the analysis can be greatly reduced. In one or more exemplary methods, the preparation time can be less than 60 seconds, 55 seconds, 50 seconds, 45 seconds, 40 seconds, 35 seconds, 30 seconds, 25 seconds, 20 seconds, 15 seconds, 10 seconds, or 5 seconds. In one or more exemplary methods, the preparation time can be greater than 60 seconds, 55 seconds, 50 seconds, 45 seconds, 40 seconds, 35 seconds, 30 seconds, 25 seconds, 20 seconds, 15 seconds, 10 seconds, or 5 seconds. Further, the need for mixing and/or incubation may be reduced in turn reducing the risk of damaging or otherwise destroying the blood sample, e.g. by activating platelets in the blood sample.

Also disclosed herein is a system for performing (e.g., operating) any one of the methods disclosed herein. The system can include one or more modules (e.g., devices, systems) for the above components. For example, the system can include an inlet module. The system can include a reagent module. The system can include a mixer module. The system can include an incubation module. The mixer module and the incubation module may be the same. The system can include a hematological analysis module. The system can include one or more sensors or sensor modules. The system can include one or more optical sensors or optical sensor modules.

In one or more exemplary methods, a system for continuously monitoring and adjusting a reagent for point of care hematology can be configured to perform a method according to the disclosure. The system may include a controller. The system may include a reservoir.

The reservoir may be configured for accommodating a reagent. For example, the reagent can be the first reagent and/or the second reagent. The system can further include a cell measurement component. The cell measurement component can accommodate a blood sample. The blood sample may be the initial mixture, the first mixture, etc.

As discussed, the different modules may be operated manually or automatically. For example, the modules may be interconnected electronically, such as to a computer system or controller. The computer system may include wired or wireless connections to the different modules. The computer system may include a processer. The computer system may include one or more controllers. The computer system may control the different modules according to the combination scheme. Thus, in one or more exemplary methods, a user may input the blood sample and receive the hematological results without needing to take any further action.

In one or more exemplary methods, the computer system can include a computer program product. The computer program product can include a non-transitory computer readable medium. The non-transitory computer readable medium can have thereon a computer program. The computer program can include program instructions. The computer program can be loadable into a data processing unit. The computer program can be configured to cause execution of the steps, processes, and/or modules discussed above. For example, when the computer program is run by a data processing unit.

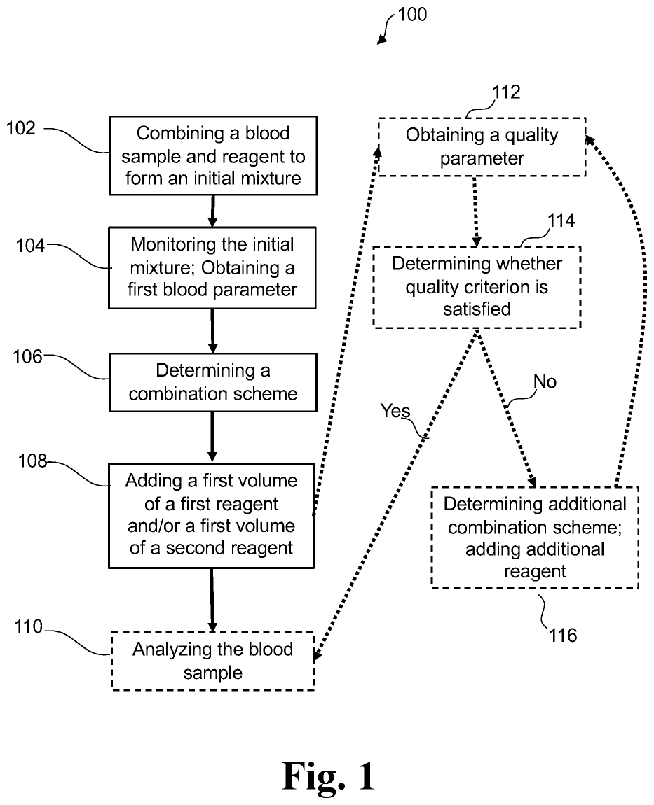

FIG. 1 illustrates an exemplary method of the disclosure. As shown, the method 100 is for preparing a blood sample, specifically by monitoring and adjusting reagent levels. The method 100 can be used for hematological analysis, as discussed above. Further, the method 100 can be used at a point-of-care for a patient.

As shown, the method 100 can include combining a blood sample with an initial volume of a first reagent to form an initial mixture 102. The combining 102 may or may not include an active mixing and/or incubation. The first reagent can be a staining and/or lysing reagent. The initial mixture may undergo preparations according to an initial combination scheme.

Further, the method 100 can include monitoring the initial mixture, which can include obtaining a first blood parameter 104 of a first blood component of the initial mixture. The first blood parameter can be any number of parameters as discussed herein, such as a red blood cell content and/or a white blood cell content and/or a platelet content and/or a lipid content.

The monitoring the initial mixture can include, but is not limited to, monitoring via image capturing and/or via an optical diffraction tomography and/or via a turbidity sensor and/or via a modified liquid sensor and/or via a porous mirror and/or via a spectrophotometer and/or via an electro/impedance measuring unit.

The obtaining the first blood parameter of the first blood component of the initial mixture can include obtaining image data and/or sensor data of the initial mixture and determining the first blood parameter based on the image data and/or the sensor data.

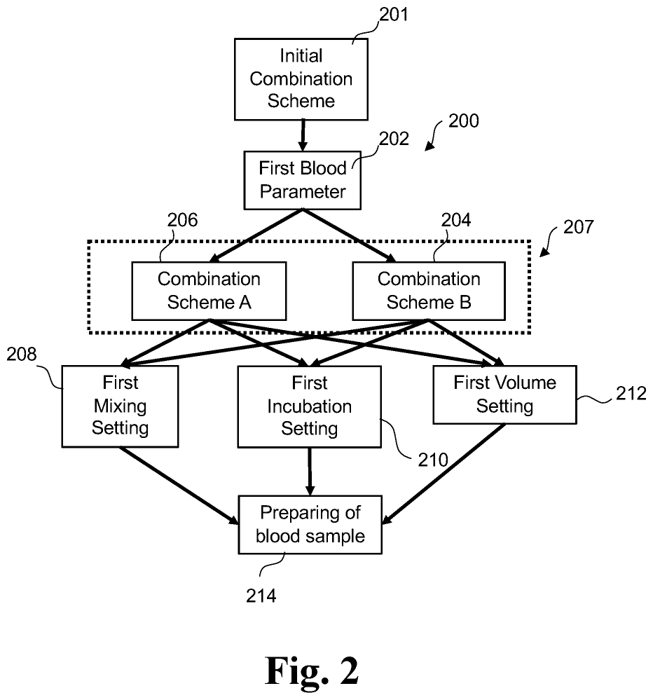

Following, the method 100 can include determining a first combination scheme 106. The first combination scheme can be based on the first blood parameter received from the monitoring and obtaining the first blood parameter 104. The first combination scheme may be selected from a number of different combination schemes, such as shown in FIG. 2. The proper combination scheme can prepare the initial mixture in a way that fast and accurate hematological analysis can be performed.

Based on the first combination scheme, the method 100 can then include adding 108 a first volume of a first reagent and/or a first volume of a second reagent to the initial mixture according to the first combination scheme for provision of a first mixture. The first mixture can be prepared in many different ways, such as discussed in detail above. This can include reagent levels, incubation settings, mixing settings, etc.

Optionally, after the preparing the first mixture by adding 108, the method 100 can include analyzing 110 the first mixture. Specifically, hematological analysis can be performed on the first mixture. As an example, a complete blood count (CBC) analysis of the first mixture can be performed after preparing the blood sample, adding the reagent, and incubating the blood sample and/or mixing the blood sample to form the first mixture. Advantageously, following method 100, full red blood cell lysis and strong white blood cell and/or platelet staining can be achieved.

Optionally, the method 100 can include obtaining 112 a quality parameter of the first mixture. For example, the quality parameter can include amount and/or percentage of blood cells that have been lysed or not lysed in the first mixture. The quality parameter can be compared to a quality criterion in determining 114 whether the criterion satisfies the quality criterion. If the quality criterion is satisfied, the method 100 can move to analyzing 110 the first mixture/blood sample. If the quality criterion is not satisfied, the method 100 can include determining a second combination scheme and adding a second volume of the first reagent and/or a second volume of the second reagent to the first mixture according to the second combination scheme for provision of a second mixture 116. The quality parameter of the second mixture can then be obtained again 112, and the check of step 114 can be repeated. This process of steps 112, 114, and 116 can be repeated any number of times until the quality criterion is satisfied and/or until a maximum number of iterations has been performed.

FIG. 2 illustrates a process that can be encompassed within method 100. As shown, the process 200 can start with the initial combination scheme 201 of an initial mixture. After performing the initial combination scheme 201 on the initial mixture, the process 200 can monitor and obtain a first blood parameter 202 (compare with step 104 of FIG. 1). Based on the first blood parameter, a particular first combination scheme can be determined. For example, a first combination scheme can be selected from a list of combination schemes 207. The list of combination schemes 207 can include combination scheme A 206 and a combination scheme B 204. Of course, the list of combination schemes 207 may include significantly more combination schemes than shown in FIG. 2.

As shown, each of the combination schemes 204/206, as well as the initial combination scheme 201, can include different settings, such as a first mixing setting 208, a first incubation setting 210, and/or a first volume setting 212. For example, the first incubation setting 210 of combination scheme A 206 may include a longer incubation time than the first incubation setting 210 of combination scheme B 204. Thus, if a longer incubation time may be advantageous for preparing the initial mixture/blood sample based on the first blood parameter, combination scheme A 206 may be determined and selected. This can provide proper preparing 214 of the initial mixture/blood sample, allowing for improved hematological analysis. The preparing 214 of the initial mixture/blood sample can include adding a first volume of the first reagent and/or a first volume of a second reagent.

In particular, the combination schemes 204/206 include a first volume setting 212 indicative of a volume of the reagent, and the preparing 214 the first mixture/blood sample can include adding a first volume of reagent to the initial mixture in a volume according to the first volume setting 212.

Further, the combination schemes 204/206 optionally include a first mixing setting 208 indicative of a mixing configuration for a mixing of the initial mixture and reagent, and the preparing 214 the initial mixture includes mixing the initial mixture and reagent according to the first mixing setting 208. The first mixing setting 208 includes one or more of a number of mixing cycles, a flow velocity, and a mixing time.

Additionally, the combination schemes 204/206 optionally include a first incubation setting 210 indicative of an incubation configuration, such as incubation time and/or incubation temperature, for the initial mixture/blood sample, and the preparing the blood sample 214 includes incubating the blood sample according to the first incubation setting 210. The first incubation setting 210 includes one or both of an incubation temperature and an incubation time.

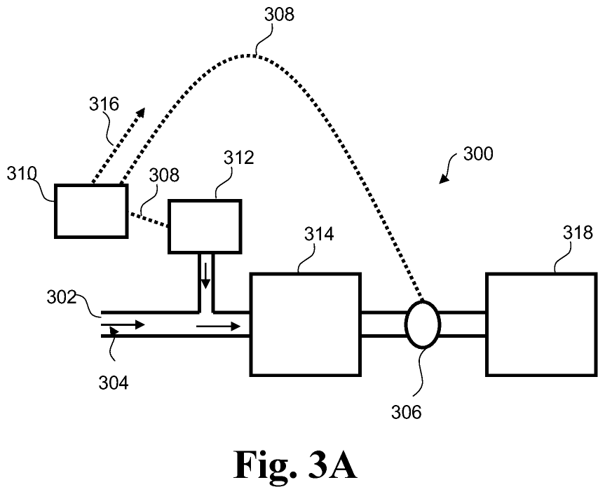

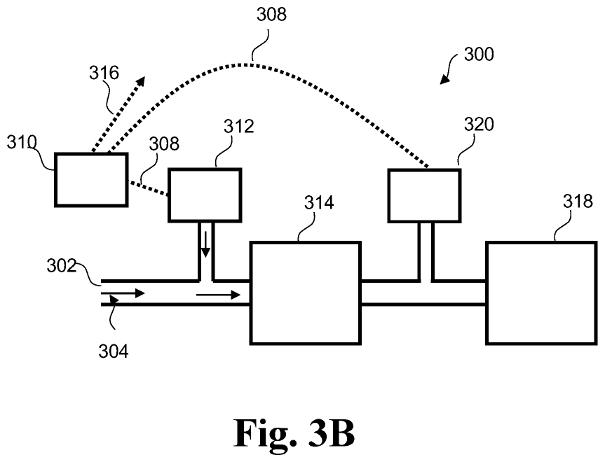

FIG. 3A illustrates a schematic of a system that can utilize any or all of the above-disclosed methods herein. As shown, the system 300 can include an inlet (e.g., inlet module) 302. The inlet 302 can be configured to receive a blood sample. The inlet 302 can be connected to a fluid pathway 304 for providing the blood sample through the system 300.

As shown, the blood sample may be combined with an initial volume of reagent from reservoir 312 in fluid connection with the fluid pathway 304 to form an initial mixture. Once combined, the initial mixture may continue along the fluid pathway 304 and pass through the mixer-incubator 314, such as according to the initial combination scheme.

Following, the mixer-incubator 314, the initial mixture may pass through a first sensor 306. The first sensor 306 may be any number of sensors, such as a conductivity/impedance sensor or an absorbance/transmittance/refractive index sensor. The first sensor 306 may obtain image data and/or sensor data of the initial mixture. The first sensor 306 may be used to obtain the first blood parameter discussed herein. The first sensor 306 may perform a blood gas analysis and/or a basic metabolic panel analysis. The first sensor 306 may be an optical sensor.

In the system shown in FIG. 3A, the first sensor 306 may include an electrical connection 308 to a computer system 310, such as including a controller. The computer system 310 may then determine a combination scheme that can be used for preparing the sample. The computer system then may be connected to provide data to a reservoir 312 configured to accommodate a reagent, and a mixer-incubator 314 configured to mix and/or incubate. Further, the computer system 310 may output 316 the data, such as a first blood parameter and/or a selected combination scheme and/or a first reagent indicator indicative of a first combination scheme. For example, the output 316 may include a combination indicator indicative of the combination scheme. This may be output to a display, such as a monitor and/or tablet and/or phone, either through a wireless or wired connection.

The computer system 310 may determine a second combination scheme, for example that more reagent, or a second reagent, should be added into the initial mixture to further lyse and/or stain the mixture. Thus, the mixture may reverse direction and pass through the mixer-incubator 314, such as according to the first combination scheme. Alternatively, the mixture may go around the mixture-incubator 314 and thus only pass through the mixer-incubator in one direction.

Following, the computer system 310 may control how much additional reagent is added to the fluid pathway 304 to mix with the initial mixture based on the combination scheme to form a first mixture. Further, the computer system 310 may control mixing settings and incubation settings of the mixer-incubator 314. As shown, the first mixture may pass through the mixer-incubator 314 again along the fluid pathway according to the first combination scheme. The first sensor 306 may again provide a blood parameter to the computer system 310, which can determine whether additional reagent is needed or whether the first mixture can continue.

Once the first mixture has been prepared according to the first combination scheme, the first mixture can pass on to the blood analyzer 318, which can perform hematological analysis on the first mixture. The first mixture can be added into a cell measurement component for use in the blood analyzer 318. After the first mixture has been analyzed, it can be discarded.

FIG. 3B illustrates an alternate configuration of the system 300 as shown in FIG. 3A. Thus, unless mentioned, any and/or all components discussed with respect to FIG. 3A can be included in FIG. 3B. As shown, instead of using the first sensor 306, the system 300 can include a separate flow channel to a blood gas analyzer 320. The blood gas analyzer 320 can provide the first blood parameter as discussed herein. Thus, the blood sample is split in two, and the blood gas analyzer 320 can inform the computer system 310 and/or the blood analyzer 318. As shown, the blood gas analyzer 320 can be located after the mixer-incubator 314.

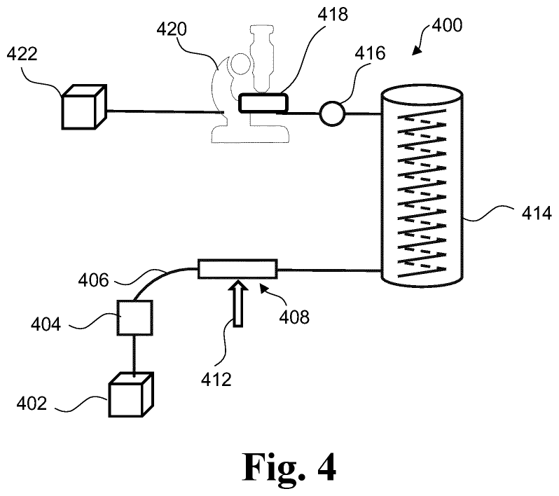

FIG. 4 illustrates a more detailed example system. As shown, the system 400 can include blood sample aspiration and/or pre-heating 402. The blood sample can then pass through an inlet 404 to a fluid pathway 406. The blood sample can approach a T-junction 408, which can include a liquid sensor. The T-junction 408 can be in fluid communication with a dosing pump 412, which can provide reagent for provision to the initial mixture.

The initial mixture can then move through the helical mixer 414 to actively mix the first mixture, such as in accordance with the initial combination scheme. As discussed, the initial mixture can pass through the helical mixer 414 one or more times. The helical mixer 414 may also incubate the initial mixture.

Once mixed and incubated according to the combination scheme, the initial mixture may pass through a first liquid sensor 416. The first liquid sensor 416 may be a first optical sensor. The first liquid sensor 416 can obtain the first blood parameter, and the first combination scheme can be determined. The first liquid sensor 410 may be a hematocrit (Hct) sensor.

Based on the first combination scheme, the initial mixture may pass back through the helical mixer 414, and a particular additional volume of reagent can be added to the initial mixture through the dosing pump 412 to form a first mixture, such as according to the first combination scheme. The first mixture may then pass through the helical mixer 414 and pass through the first liquid sensor 416 again, such as according to the first combination scheme. If the results are satisfactory, the first mixture may move on to cuvette 418.

The first mixture can then be passed to a reusable cuvette 418, where it can be analyzed in a blood analyzer 420. This can be, for example, an optical microscope, and hematological analysis can be performed. Once the analysis is finished, the first mixture can pass to a waste module 422.

Examples of methods for monitoring and adjusting reagent for hematology according to the disclosure are set out in the following items:

Item 1. Method of preparation of a blood sample with a reagent for hematology, the method comprising:
combining a blood sample with an initial volume of a first reagent for provision of an initial mixture;
monitoring the initial mixture, wherein monitoring the initial mixture comprises obtaining a first blood parameter of a first blood component of the initial mixture;
determining a first combination scheme based on the first blood parameter; and
adding a first volume of the first reagent and/or a first volume of a second reagent to the initial mixture according to the first combination scheme for provision of a first mixture.

Item 2. Method according to Item 1, wherein the method further comprises outputting a first reagent indicator indicative of the first combination scheme.

Item 3. Method according to any one of the preceding Items, wherein the obtaining the first blood parameter of the first blood component of the initial mixture comprises obtaining image data and/or sensor data of the initial mixture and determining the first blood parameter based on the image data and/or the sensor data.

Item 4. Method according to any one of the preceding Items, wherein the monitoring the initial mixture comprises monitoring via image capturing and/or via an optical diffraction tomography and/or via a turbidity sensor and/or via a modified liquid sensor and/or via a porous mirror and/or via a spectrophotometer and/or via an electro/impedance measuring unit.

Item 5. Method according to any one of the preceding Items, wherein the combining comprises mixing the blood sample with the initial volume of the first reagent for provision of the initial mixture.

Item 6. Method according to any one of the preceding Items, wherein the first blood parameter is indicative of a red blood cell content in the initial mixture and/or a white blood cell content in the initial mixture and/or a platelet content in the initial mixture and/or a lipid content in the initial mixture.

Item 7. Method according to any one of the preceding Items, wherein the initial volume of the first reagent is in the range from 0.5 to 20 times a volume of the blood sample in the initial mixture.

Item 8. Method according to any one of the preceding Items, wherein the first reagent is a staining and/or lysing reagent.

Item 9. Method according to any one of the preceding Items, wherein the second reagent is a staining and/or lysing reagent.

Item 10. Method according to any one of the preceding Items, wherein the first combination scheme is configured to achieve full red blood cell lysis and strong white blood cell and/or platelet staining.

Item 11. Method according to any one of the preceding Items, the method further comprising:
obtaining a quality parameter of the first mixture; and
determining whether the quality parameter satisfies a quality criterion, wherein:
   in accordance with a determination that the quality criterion is satisfied, performing a complete blood count (CBC) analysis of the first mixture; and
   in accordance with a determination that the quality criterion is not satisfied, determining a second combination scheme and adding a second volume of the first reagent and/or a second volume of the second reagent to the first mixture according to the second combination scheme for provision of a second mixture.

Item 12. Method according to Item 11, wherein the quality parameter is indicative of a degree of red blood cell lysis and wherein the quality criterion is satisfied if the quality parameter is larger than a first lysis threshold.

Item 13. Method according to Item 11, wherein the quality parameter is indicative of a degree of white blood cell lysis and/or platelet lysis and wherein the quality criterion is satisfied if the quality parameter is larger than a second lysis threshold.

Item 14. A computer program product comprising a non-transitory computer readable medium, having thereon a computer program comprising program instructions, the computer program being loadable into a data processing unit and configured to cause execution of the steps according to any of Items 1 through 13 when the computer program is run by the data processing unit.

Item 15. A system for continuously monitoring and adjusting a reagent for point of care hematology, the system comprising:
a controller;
a reservoir for accommodating a reagent; and
a cell measurement component for accommodating a blood sample;
wherein the system is configured to perform the method according to any one of the preceding Items.

In a first aspect of the invention, the blood analyzer disclosed in present disclosure is configured to analyze biological fluids, such as, e.g., human, animal, mammalian blood, and/or cell cultures. Moreover, in said further aspect the blood analyzer is substituted by and/or comprises a biological fluid analyzer, such as, e.g., a blood analyzer and/or a cell culture analyzer.

In the first aspect, any disclosed blood sample may be substituted by and/or comprise a biological fluid sample, such, e.g., as a human blood sample, an animal blood sample, a mammalian blood sample, and/or a cell culture sample.

In the first aspect, any disclosed prepared blood sample may be substituted by and/or comprise a prepared biological fluid sample, such, e.g., as a prepared human blood sample, a prepared animal blood sample, a prepared mammalian blood sample, and/or a prepared cell culture sample.

In the first aspect, the method of preparation of a blood sample with a reagent for hematology may be a method of preparation of a biological fluid sample with a reagent for biological fluid analysis.

In the first aspect, any disclosed blood parameter may be substituted by and/or comprise a biological fluid parameter, such as human blood parameter, an animal blood parameter, a mammalian blood parameter, and/or a cell culture parameter.

In some embodiments of the first aspect, the cell culture comprises a culture of cells derived from multicellular eukaryotes, such as, e.g., mammalian cells, animal cells, and/or human cells. In some embodiments, the cell culture comprises a culture of cells grown from plant tissue culture, fungal culture, and/or microbiological culture (of microbes).

In the first aspect, a cell may therefore be a mammalian cell, an animal cell, a human cell, a plant tissue cultured cell, a fungal cultured cell, or a microbiologically cultured cell.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Furthermore, the labelling of a first element does not imply the presence of a second element and vice versa.

It may be appreciated that FIGS. 1-4 comprise some modules or operations which are illustrated with a solid line and some modules or operations which are illustrated with a dashed line. The modules or operations which are comprised in a solid line are modules or operations which are comprised in the broadest example embodiment. The modules or operations which are comprised in a dashed line are example embodiments which may be comprised in, or a part of, or are further modules or operations which may be taken in addition to the modules or operations of the solid line example embodiments. It should be appreciated that these operations need not be performed in order presented.

Furthermore, it should be appreciated that not all of the operations need to be performed. The exemplary operations may be performed in any order and in any combination.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the exemplary embodiments may be implemented at least in part by means of both hardware and software, and that several “means”, “units” or “devices” may be represented by the same item of hardware.

The various exemplary methods, devices, and systems described herein are described in the general context of method steps processes, which may be implemented in one aspect by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform specified tasks or implement specific abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Although features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications, and equivalents.

### LIST OF REFERENCES

100 method of preparing a blood sample for hematological analysis
102 combining a blood sample with an initial volume of a first reagent
104 monitoring the initial mixture; obtaining a first blood parameter
106 determining a combination scheme
108 adding a first volume of a first reagent and/or a first volume of a second reagent
110 optionally analyzing a blood sample
112 obtaining a quality parameter
114 determining whether the quality parameter satisfies a quality criterion
116 determining an additional combination scheme and adding an additional volume of reagent
200 process of preparing a blood sample
201 initial combination scheme
202 first blood parameter
204 combination scheme a
206 combination scheme b
207 list of combination schemes
208 first mixing setting
210 first incubation setting
212 first volume setting
214 preparing blood sample
300 system
302 inlet
304 fluid pathway
306 first sensor
308 electrical connection
310 computer system
312 reservoir
314 mixer-incubator
316 output
318 blood analyzer
320 blood gas analyzer
400 system
402 sample aspiration and/or pre-heating
404 inlet
406 fluid pathway
408 T-junction
412 dosing pump
414 helical mixer
416 first liquid sensor
418 reusable cuvette
420 blood analyzer
422 waste module

1. A method of preparing a blood sample with a reagent for hematology, the method comprising:
combining a blood sample with an initial volume of a first reagent for provision of an initial mixture;
monitoring the initial mixture, wherein monitoring the initial mixture comprises obtaining a first blood parameter of a first blood component of the initial mixture;
determining a first combination scheme based on the first blood parameter; and
adding a first volume of the first reagent and/or a first volume of a second reagent to the initial mixture according to the first combination scheme for provision of a first mixture.

2. The method according to claim 1, wherein the method further comprises outputting a first reagent indicator indicative of the first combination scheme.

3. The method according to claim 1, wherein the obtaining the first blood parameter of the first blood component of the initial mixture comprises obtaining image data and/or sensor data of the initial mixture and determining the first blood parameter based on the image data and/or the sensor data.

4. The method according to any claim 1, wherein the monitoring the initial mixture comprises monitoring via image capturing and/or via an optical diffraction tomography and/or via a turbidity sensor and/or via a modified liquid sensor and/or via a porous mirror and/or via a spectrophotometer and/or via an electro/impedance measuring unit.

5. The method according to any claim 1, wherein the combining comprises mixing the blood sample with the initial volume of the first reagent for provision of the initial mixture.

6. The method according to any claim 1, wherein the first blood parameter is indicative of a red blood cell content in the initial mixture and/or a white blood cell content in the initial mixture and/or a platelet content in the initial mixture and/or a lipid content in the initial mixture.

7. The method according to any claim 1, wherein the initial volume of the first reagent is in the range from 0.5 to 20 times a volume of the blood sample in the initial mixture.

8. The method according to any claim 1, wherein the first reagent is a staining and/or lysing reagent.

9. The method according to any claim 1, wherein the second reagent is a staining and/or lysing reagent.

10. The method according to any claim 1, wherein the first combination scheme is configured to achieve full red blood cell lysis and strong white blood cell and/or platelet staining.

11. The method according to any claim 1, the method further comprising:
obtaining a quality parameter of the first mixture; and
determining whether the quality parameter satisfies a quality criterion, wherein:
in accordance with a determination that the quality criterion is satisfied, performing a complete blood count (CBC) analysis of the first mixture; and
in accordance with a determination that the quality criterion is not satisfied, determining a second combination scheme and adding a second volume of the first reagent and/or a second volume of the second reagent to the first mixture according to the second combination scheme for provision of a second mixture.

12. The method according to claim 11, wherein the quality parameter is indicative of a degree of red blood cell lysis and wherein the quality criterion is satisfied if the quality parameter is larger than a first lysis threshold.

13. The method according to claim 11, wherein the quality parameter is indicative of a degree of white blood cell lysis and/or platelet lysis and wherein the quality criterion is satisfied if the quality parameter is larger than a second lysis threshold.

14. A computer program product comprising a non-transitory computer readable medium, having thereon a computer program comprising program instructions, the computer program being loadable into a data processing unit and configured to cause execution of the steps according to claim 1 when the computer program is run by the data processing unit.

15. A system for continuously monitoring and adjusting a reagent for point of care hematology, the system comprising:
a controller;
a reservoir for accommodating a reagent; and
a cell measurement component for accommodating a blood sample;
wherein the system is configured to perform the method according to claim 1.
